(19) United States
(12) Patent Application Publication
(10) Pub. No.: US 2007/0254306 A1
(43) Pub. Date: Nov. 1, 2007

(54) Method of determining genetic predisposition for deficiency in health functions using SNP analysis

(76) Inventor: Vincent C. Giampapa, Little Falls, NJ (US)

Correspondence Address: MELVIN K. SILVERMAN, 500 WEST CYPRESS CREEK ROAD, SUITE 500, FT. LAUDERDALE, FL, 33309, US

(21) Appl. No.: 11/796,966

(22) Filed: Apr. 30, 2007

Related U.S. Application Data
(60) Provisional application No. 60/796,423, filed on May 1, 2006.

(51) Int. Cl. C12Q 1/68; G06F 19/00

(52) U.S. Cl. 435/6; 702/20

(57) ABSTRACT

A method of determining genetic predisposition for deficiency in a specific health function of a person using single nucleotide polymorphism (SNP) analysis is provided. The method includes performing a SNP genotyping assay of three or more genes using a sample obtained from the person, obtaining a SNP panel which includes predetermined identifier SNPs, comparing the SNP panel with a predetermined criterion defining the genetic predisposition for deficiency in a specific health function; and reporting presence of genetic predisposition for deficiency in this health function if the SNP panel meets the predetermined criterion. The health function includes glycation, inflammation, DNA methylation, oxidation or DNA repair.

### CROSS REFERENCE TO RELATED APPLICATION

This application claims the benefit under 35 USC 119 (e) of the provisional patent application Ser. No. 60/796,423, filed on May 1, 2006, which is herein incorporated by reference in its entirety.

### FIELD OF THE INVENTION

The present invention relates to methods of determining genetic predisposition for deficiency in health functions, including glycation, inflammation, DNA methylation, oxidation and DNA repair, of a person using SNP analysis.

### BACKGROUND OF THE INVENTION

Single nucleotide polymorphisms or SNPs are DNA sequence variations that occur when a single nucleotide (A, T, C, or G) in the genome sequence is altered. For example a SNP might change the DNA sequence MGGC™ to ATGGCTAA. For a variation to be considered a SNP, it must occur in at least 1% of the population. SNPs, which make up about 90% of all human genetic variation, occur every 100 to 300 bases along the 3-billion-base human genome. Two of every three SNPs involve the replacement of cytosine (C) with thymine (T). SNPs can occur in both coding (gene) and noncoding regions of the genome. Many SNPs have no effect on cell function, however others could predispose people to disease or influence their response to a drug.

Although more than 99% of human DNA sequences are the same across the population, variations in DNA sequence can have a major impact on how humans respond to disease; environmental insults such as bacteria, viruses, toxins, and chemicals; drugs and other therapies. SNPs are also evolutionarily stable, not changing much from generation to generation.

With adequate information on the interaction between specific genetic polymorphisms, diet and risks in developing certain clinical conditions, it becomes possible to provide individuals with dietary guidance tailored according to their genotype. This raises the possibility of providing individualized nutritional advice on the basis of genotype. For example, women who are carriers of the A allele should increase their intake of polyunsaturated fatty acids (PUFAs) to increase HDL-cholesterol concentrations and reduce the risk in cardiovascular diseases, whereas G/G women should receive the opposite advice. As another example, it is also known that the expression and activity of cSHMT gene is regulated robustly by several nutrients, including folate, zinc, and ferritin.

The most evident demonstration of the effects of dietary nutrition on genetic functions of a mammal is the caloric restriction studies and caloric restriction mimic studies in mice. It has reported that metformin, a drug used to treat diabetes, can mimic many of the changes in gene expression found in calorically-restricted mice, which live much longer, healthier lives than normally fed mice.

It is known that glycation, inflammation, DNA methylation, oxidation, and DNA repair are five important biological processes or functions in human. Maintaining normal functions in these processes directly affects an individual's overall health and aging.

DNA methylation is the covalent addition of a methyl group to the 5-carbon position of cytosine, predominantly within cytosine guanine dinucleotides (CpG). It is known that DNA methylation is an important epigenetic mechanism of transcriptional control. Recent studies have demonstrated that methyl insufficiency and/or abnormal DNA methylation likely have significant roles in the development of several pathologies including birth defects, cancer, diabetes, heart disease and neurological disorders. The evidence that DNA methylation is influenced by diet and dietary factors arises from both preclinical and clinical studies. For example, folate depletion resulted in lymphocyte DNA hypomethylation in postmenopausal women, which was reversed following folate repletion. Dietary factors that have been reported to have impacts on DNA methylation processes include folate, vitamin B₁₂ (cobalamin), vitamin B₆ (pyridoxine), vitamin B₂ (riboflavin), methionine, choline and alcohol.

Glycation is the result of a sugar molecule, such as fructose or glucose, bonding to a protein or lipid molecule without the controlling action of an enzyme. Over time, the sugar moieties bound to the glycated proteins are chemically modified to become molecular structures called Advanced Glycation Endproducts (A.G.E.s). A.G.E.s can interfere with the proper functioning of the proteins to which they are attached. Furthermore, some of the A.G.E.s form covalent crosslinks with adjacent protein strands. This crosslinking stiffens tissues which were formerly flexible or elastic. Glycation changes the shape and properties of proteins. Crosslinking reduces the flexibility, elasticity, and functionality of the proteins. Furthermore, the chemical modifications of glycation and crosslinking can initiate harmful inflammatory and autoimmune responses. Glycation and crosslinking have been implicated as strong contributors to many progressive diseases of aging, including vascular diseases (such as atherosclerosis, systolic hypertension, pulmonary hypertension, and poor capillary circulation), erectile dysfunction, kidney disease, stiffness of joints and skin, arthritis, cataracts, retinopathy, neuropathy, Alzheimer's Dementia, impaired wound healing, urinary incontinence, complications of diabetes, and cardiomyopathies (such as diastolic dysfunction, left ventricular hypertrophy, and congestive heart failure).

Furthermore, it has been founded that insulin sensitivity and glycation are closely related to each other; increase of insulin sensitivity in an individual reduces the glycation process. Studies have indicated a tendency for mature adults to lose sensitivity to insulin. Sedentary lifestyle, obesity, and a diet low in fiber and chromium and high in sugars, all contribute to decreased insulin sensitivity. It has been reported that vanadium and chromium, when ingested, have properties that closely mimic, as well as enhance, many of the physiological effects of insulin. In this respect, it has been found that these elements serve to both increase the effectiveness and enhance the anabolic effects of insulin.

On the other hand, free radicals promote beneficial oxidation that produces energy and kills bacterial invaders, but in excess through accumulation, they can disturb cell structure, resulting in cellular damage in protein, fat and DNA molecules. Giampapa “The Basic Principles and Practice of Anti-Aging Medicine & Age Management”, Self-published, Nespift Graphics Inc., 2003, provides a review on aging, with a focus on understanding the oxidative stress and gene expression. More specifically, among the inflammatory chain activities triggered by free radicals, it is known that the transcription factors NF-kB and activator protein 1 (AP-1) are activated by free radicals and pro-inflammatory cytokines, which are generated by free radical activity. NF-kB and AP-1 play critical roles in the regulation of pro-inflammatory cytokines and related proteins, and collagen-digesting enzymes. Because free radicals and pro-inflammatory cytokines activate the transcription factors, the result is a self-reinforcing pro-inflammatory cycle. This pro-inflammatory cycle, together with the free radical damage to the cell membrane, produce intracellular free radicals that oxidize the polyunsaturated fatty acids rich membranes surrounding the cytoplasm's organelles, mitochondria and nucleus. The pro-inflammatory cycle initiated by oxidative stress on the cell membrane gradually weakens the most basic functions of cells.

Antioxidant supplements have been used in enhancing an individual's resistance to oxidative damages. These include vitamin A and beta carotene, vitamin C, vitamin E, selenium, coenzyme Q-10, certain vitamin B complex, and alpha lipoic acid.

It is known that DNA repair is a process constantly operating in cells. It is essential to survival because it protects the genome from damage and harmful mutations. In human cells, both normal metabolic activities and environmental factors (such as UV rays) can cause DNA damage, resulting in as many as 500,000 individual molecular lesions per cell per day. These lesions cause structural damage to the DNA molecule, and can dramatically alter the cell's way of reading the information encoded in its genes. Consequently, the DNA repair process must be constantly operating, to correct rapidly any damage in the DNA structure.

As cells age, however, the rate of DNA repair decreases until it can no longer keep up with ongoing DNA damage. The cell then suffers one of three possible fates: an irreversible state of dormancy, known as senescence; cell suicide, also known as apoptosis or programmed cell death; carcinogenesis, or the formation of cancer. Most cells in the body first become senescent. Then, after irreparable DNA damage, apoptosis occurs. In this case, apoptosis functions as a “last resort” mechanism to prevent a cell from becoming carcinogenic and endangering the organism.

When cells become senescent, alterations in biosynthesis and turnover cause them to function less efficiently, which inevitably causes disease. The DNA repair ability of a cell is vital to the integrity of its genome and thus to its normal functioning and that of the organism. Many genes that were initially shown to influence lifespan have turned out to be involved in DNA damage repair and protection.

Recent scientific studies, particularly in pharmacogenomic studies in the last few years, have identified various of SNPs involved in the biological functions or processes described above. However, the information has not be utilized in a systematic manner for determining an individual's genetic predisposition for deficiency in specific biological function or process and providing treatment nutriceutically to improve the biological function or process, or decrease the likelihood of the individual in developing clinical diseases, and reduce the rate of aging due to such a deficiency.

### SUMMARY OF THE INVENTION

In one aspect, the present invention is directed to a method of determining genetic predisposition for deficiency in a specific health function of a person using single nucleotide polymorphism (SNP) analysis. The method comprises obtaining a sample from a person; performing a SNP genotyping assay of three or more genes using the sample; obtaining a SNP panel comprising predetermined identifier SNPs; comparing the SNP panel with a predetermined criterion defining the genetic predisposition for deficiency in a specific health function; and reporting presence of genetic predisposition for deficiency in the health function if the SNP panel meets the predetermined criterion. The health function includes glycation, inflammation, DNA methylation, oxidation or DNA repair.

More specifically, in one embodiment, the method is directed to determine genetic predisposition for deficiency in glycation of a person, wherein the genes analyzed in the SNP genotyping assay include ACE, LEPR, and PPARG, and the predetermined glycation identifier SNPs include rs4646994, rs1137101, rs1137100, and rs3856806, expressed by dbSNP ID. The method further includes providing a nutritional supplement specific to reduce glycation if the genetic predisposition of deficiency in glycation is identified.

In a further embodiment, the method is directed to determine genetic predisposition for deficiency in inflammation of a person, wherein the genes analyzed in the SNP genotyping assay include ICAM1, IL6, NFKB1, NOS3, NPY, PPARA, and TNF; and the predetermined inflammation identifier SNPs include rs5498, rs1800795, rs28362491, rs1799983, rs16139, rs1800206, and rs1800629, expressed by dbSNP ID. The method further includes providing to the person a nutritional supplement specific to decrease the likelihood in developing inflammation if the genetic predisposition for deficiency in inflammation is identified.

In another embodiment, the method is directed to determine genetic predisposition for deficiency in DNA methylation of a person, wherein the genes analyzed in the SNP genotyping assay include APOC3, CETP, and MTHFR; and the predetermined methylation identifier SNPs include rs2542051, gs47114, rs5882, rs708272, rs1801131, and rs1801133, expressed by dbSNP ID. The method further includes providing to the person a nutritional supplement specific to improve DNA methylation if the genetic predisposition of deficiency in DNA methylation is identified.

In yet a further embodiment, the method is directed to determine genetic predisposition for deficiency in oxidation of a person, wherein the genes analyzed in the SNP genotyping assay include AGER, CAT, CYP2D6, NQO1, SOD2, and SOD3; and the predetermined oxidation identifier SNPs include rs3134940, rs2070600, rs1001179, rs1065852, rs1800566, rs1799725, and rs1799895, expressed by dbSNP ID. The method further includes providing to the person a nutritional supplement specific to enhance resistance to oxidative damages if genetic predisposition for deficiency in oxidation is identified.

In yet another embodiment, the method is directed to determine genetic predisposition for deficiency in DNA repair of a person, wherein the genes analyzed in the SNP genotyping assay include ERCC2, OGG1, PARP1/ADPRT, and XRCC1; and the predetermined DNA repair identifier SNPs include rs1799793, rs13181, rs1052133, rs1136410, rs25489, and rs25487, expressed by dbSNP ID. The method further includes providing to the person a nutritional supplement specific to improve said DNA repair if genetic predisposition for deficiency in DNA repair is identified.

In a further aspect, the present invention provides a determinant SNP panel for determining genetic predisposition for deficiency in health functions of a person, which comprises a glycation SNP panel comprising predetermined glycation identifier SNPs; an inflammation SNP panel comprising predetermined inflammation identifier SNPs; a methylation SNP panel comprising predetermined methylation identifier SNPs; an oxidation SNP panel comprising predetermined oxidation identifier SNPs; and a DNA repair SNP panel comprising predetermined DNA repair identifier SNPs.

### DETAILED DESCRIPTION OF THE INVENTION

In one embodiment, the present invention provides a method of determining the genetic predisposition for deficiency in health functions of a person using single nucleotide polymorphism (SNP) analysis. More specifically, the method comprises the steps of: obtaining a sample from a person; performing a SNP genotyping assay of three or more genes using the sample; obtaining a SNP panel which includes predetermined identifier SNPs; comparing the SNP panel with a predetermined criterion defining the genetic predisposition specific to a deficient health function; and reporting presence of genetic predisposition for deficiency in the health function if the SNP panel meets the predetermined criterion.

The term of health function used herein refers to a specific biological function or process, including glycation, inflammation, DNA methylation, oxidation, and DNA repair. The term of deficiency in a health function used herein refers to a deficient or compromised function or process in a health function as defined above due to genetic predisposition, which can further include sub-normal function or process in a health function.

The term of identifier SNP used herein refers to a single nucleotide polymorphism that has been identified being specific to a biological function or process described above, for example, methylation identifier SNPs are the SNPs being identified specifically related to DNA methylation. The term of SNP panel used herein refers to a set or a group of identifier SNPs specific to a biological function or process described above.

Suitable samples for the SNP genotyping assay include saliva, blood or blood fractions (e.g., serum, plasma, platelets, red blood cells, and white blood cells), sputum, and tissue such as skin, bone and hair. The SNP genotyping assay can be performed using existing SNP analysis methods known in the art. Various methods, such as single base extension (SBE) followed by tag-array hybridization and allele-specific primer extension, are commercially available for performing SNP genotyping assays. SNP genotyping assays are provided by various commercial laboratories. For example, customerized TagMan® SNP genotyping assays are provided by A&B Applied Biosystems, Foster City, Calif. The SNP genotyping assays for the purpose of the present invention have been performed by Genomics Collaborative, Inc., Cambridge, Mass. The sample was a swab taken in a person's mouth, which was sealed and delivered to Genomics Collaborative, Inc. for SNP genotyping assays.

Other than living condition, dietary, or environmental causes for developing diabetes, inflammatory diseases, cardiovascular diseases, cancers and accelerated aging, it is known that genetic predisposition increases an individual's likelihood for developing these diseases. More specifically, deficiency in glycation because of genetic predisposition increases the likelihood of an individual for developing various diseases, particular diabetic complications; deficiency in inflammation because of genetic predisposition increases the likelihood of an individual for developing inflammatory diseases; deficiency in DNA methylation because of genetic predisposition increases the likelihood of an individual for developing cardiovascular diseases, Alzheimer disease and brain loss; deficiency in oxidation because of genetic predisposition increases the likelihood of an individual to oxidative damages and general accelerated aging such as premature wrinkles of the skin; and deficiency in DNA repair because of genetic predisposition increases the likelihood of an individual for developing cellular abnormalities in the form of tumors, cancers as well as general accelerated aging. It is noted that the phrase of a person having a genetic predisposition for deficiency in oxidation means the person is more prone to oxidative damages, and more likely develop disorders directly or indirectly caused by oxidative damages; similarly, deficiency in glycation means the person is more prone to excess glycation; and deficiency in inflammation means the person is more prone to develop inflammatory diseases. As can be understood, these five identified health functions relate to the fundamentals of an individual's overall health, and longevity.

It has been found by the present invention that genetic predisposition for deficiency in glycation, inflammation, DNA methylation, oxidation, or DNA repair can be effectively determined by SNP analysis, more specifically can be determined by using a panel of identifier SNPs specific to a particular biological function or process. Using a panel of, or a plurality of, identifier SNPs, which covers the genetic involvements in different aspects of a biological function, the method assures the determination of the genetic predisposition for deficiency in that specific function.

In a further aspect of the present invention, the method further includes providing a nutritional supplement to a person who has been identified having a genetic predisposition for deficiency in a health function, such as in glycation or DNA repair, as described further hereinafter, thereby enhancing a specific health function and compensating deficiencies resulting from genetic predisposition. Therefore, the present invention provides nutritional intervention to individuals who are in need thereof because of their genetic predisposition, for decreasing the individual's likelihood of developing clinical conditions, or age-related diseases and increasing the quality of life and likelihood of longevity. It is believed that such an intervention can function similarly to the caloric restriction mimic approach in affecting gene expression and metabolic passways.

In one embodiment, the present invention provides a method for determining genetic predisposition for deficiency in glycation of a person. A sample can be collected from an individual by a mouth swab. A SNP genotyping assay is performed on three genes including angiotensin I converting enzyme (peptidyl-dipeptidase A) 1 (ACE), leptin receptor (LEPR), and peroxisome proliferative activated receptor, gamma (PPARG). From the SNP genotyping assay, a glycation SNP panel is obtained, which comprises predetermined glycation identifier SNPs. As shown in Table 1, the predetermined glycation identifier SNPs include insertion/deletion at dbSNP ID rs4646994 in gene ACE, polymorphism Gln 223 Arg (dbSNP ID rs1137101) in gene LEPR, polymorphism K109R (dbSNP ID rs1137100) in gene LEPR, and polymorphism C1431T (dbSNP ID rs3856806) in gene PPARG.

TABLE 1

| | | Gene | Polymorphism | | | |
|---|---|---|---|---|---|---|
| Gene Class | Gene Symbol | Location | Name | dbSNP_ID | Genotype | Rating |
| Glycation | ACE | 17q23 | Insertion/ | rs4646994 | DD | Deficient |
| | | | Deletion | | ID | Subnormal |
| | | | | | II | Normal |
| | LEPR | 1p31 | Gln 223 Arg | rs1137101 | ArgArg | Deficient |
| | | | | | GlnArg | Subnormal |
| | | | | | GlnGln | Normal |
| | | | K109R | rs1137100 | LysLys | Normal |
| | | | | | LysArg | Subnormal |
| | | | | | ArgArg | Subnormal |
| | PPARG | 3p25 | C1431T | rs3856806 | CC | Deficient |
| | | | | | CT | Normal |
| | | | | | TT | Subnormal |

It is noted that the gene and SNP information in Table 1, as well as other tables, is expressed by gene symbols, gene locations, polymorphism names and their identifications in public SNP database (dbSNP ID), and the genotypes using the standard expression used in molecular biology, and they are readily understood by one of ordinary skill in the art. The gene class used herein refers to the classification of the genes related to the five health functions described above. Gene symbol is the acronym or abbreviation corresponding to a given gene name. Genes and markers may have multiple symbols and names due to rediscovery or correlation to function following discovery.

In the first step of the determination, a rating is made based on each individual glycation identifier SNP. As illustrated in Table 1, for insertion/deletion at dbSNP ID rs4646994 in gene ACE the predetermined criterion defines that for a person having genotype II, the genetic predisposition for glycation is normal; for a person having genotype ID, the genetic predisposition for glycation is sub-normal; and for a person having genotype DD, the genetic predisposition for glycation is deficient or compromised. For polymorphism Gln 223 Arg (dbSNP ID rs1137101) in gene LEPR, the predetermined criterion defines that for a person having genotype GlnGln, the genetic predisposition for glycation is normal; for a person having genotype GlnArg, the genetic predisposition for glycation is sub-normal; and for a person having genotype ArgArg, the genetic predisposition for glycation is deficient. For polymorphism K109R (dbSNP ID rs1137100) in gene LEPR, the predetermined criterion defines that for a person having genotype LysLys, the genetic predisposition for glycation is normal; for a person having genotypes LysArg or ArgArg, the genetic predisposition for glycation is sub-normal. For polymorphism C1431T (dbSNP ID rs3856806) in gene PPARG, the predetermined criterion defines that for a person having genotype CT, the genetic predisposition for glycation is normal; for a person having genotype TT, the genetic predisposition for glycation is sub-normal; and for a person having genotype CC, the genetic predisposition for glycation is deficient.

In the second step, the method combines the rating for each glycation identifier SNP in the first step described above together, then obtains an overall rating for glycation. For example, in a 1-10 scale, the rating for normal, sub-normal and deficient can be 10, 5 and 1, respectively. In the second step, the ratings for the individual glycation identifier SNPs are added together, and an average is obtained as an overall rating for glycation. For example, a criterion is set that an overall rating of greater than 8 is defined as normal, the overall rating of between 6 and 8 is defined as sub-normal, and the overall rating below 6 is defined as deficient in genetic predisposition for glycation.

In a further embodiment, the individual glycation identifier SNPs can have different weights in the process of determining the overall rating. More specifically, a weighing factor can be assigned to each glycation identifier SNP depending on their importance on the glycation process. Therefore, in the second step a weighted average is obtained. For example, if a person's genotype of dbSNP ID rs1137101 in gene LEPR is ArgArg, statistically the likelihood of the person for developing a clinical condition, such as complications of diabetes, is substantially higher than if the person has a genotype defined as deficient in any other glycation identifier SNP described above, then the genotype ArgArg of dbSNP ID rs1137101 in gene LEPR can have more weight than others in the overall rating. For example, a weighing factor of 0.2 can be applied to the individual rating of 1 for this genotype, which then affects the overall rating on the genetic predisposition for glycation much more significantly than other glycation identifier SNPs that have a weighing factor of 1 or close to 1. The weighing factor is determined based on statistical analyses of available clinical and genetic information related to the glycation.

In a further aspect of the present invention, the method further includes providing a supplement composition to a person who has been identified having genetic predisposition for deficiency in glycation, for use as a dietary supplement to specifically reduce glycation and decrease the likelihood of developing clinical conditions related to glycation. It is known that certain nutrients, such as chromium, vanadium, and thiamin, can improve insulin sensitivity, which ultimately reduces glycation process and reduces the likelihood of a person to develop clinical conditions related to glycation. A supplement composition specific for improving insulin sensitivity and the method of use have been described in detail in a co-pending Provisional Paten Application Ser. No. 60/685,141, entitled “Supplement Composition and Method of Use for Enhancement of Insulin Sensitivity”, which is herein incorporated by reference in its entirety. Such a supplement composition is particularly suitable for the purpose of the invention. Other supplement compositions containing nutrients effective in enhancing glycation can also be used.

In a further embodiment, the present invention provides a method for determining genetic predisposition for deficiency in inflammation of a person. Similar to the method described above for determining genetic predisposition for deficiency in glycation, a panel of inflammation identifier SNPs have been identified and provided in Table 2, expressed by their gene symbols, gene locations, polymorphism names, dbSNP ID, and the genotypes. As shown, in this case a panel of seven SNPs in seven genes are used. The seven genes are intercellular adhesion molecule 1 (CD54) (ICAM 1), interleukin 6 (interferon, beta 2) (IL6), nuclear factor of kappa light polypeptide gene enhancer in B-cells 1 (p105) (NFKB1), nitric oxide synthase 3 (endothelial cell) (NOS3), neuropeptide Y (NPY), peroxisome proliferative activated receptor, alpha (PPARA), and tumor necrosis factor (TNF superfamily, member 2) (TNF). Same to that described above, individual rating, overall rating or weighted overall rating are used for determining a person's genetic predisposition for deficiency in inflammation.

In a further aspect of this embodiment, the method further includes providing a supplement composition to a person who has been identified to have genetic predisposition for deficiency in inflammation, for use as a dietary supplement to specifically enhance the person's resistance to inflammation and decrease the likelihood of developing inflammatory diseases. A supplement composition specific for improving a person's resistance to inflammation and the method of use have been described in detail in a co-pending Provisional Patent Application Ser. No. 60/693,527, entitled “Supplement Composition and Method of Use in treatment of Inflammation”, which is herein incorporated by reference in its entirety. Such a supplement composition is particularly suitable for the purpose of the invention.

TABLE 2

| | | Gene | Polymorphism | | | |
|---|---|---|---|---|---|---|
| Gene Class | Gene Symbol | Location | Name | dbSNP_ID | Genotype | Rating |
| Inflammation | ICAM1 | 19p13.3-p13.2 | K469E | rs5498 | EE | Subnormal |
| | | | | | KE | Subnormal |
| | | | | | KK | Normal |
| | IL6 | 7p21 | G-174 C | rs1800795 | CC | Deficient |
| | | | | | GC | Subnormal |
| | | | | | GG | Deficient |
| | NFKB1 | 4q24 | — | rs28362491 | II | Normal |
| | | | 94ins/delATTG | | ID | Subnormal |
| | | | | | DD | Deficient |
| | NOS3 | 7q36 | Glu298Asp | rs1799983 | AspAsp | Deficient |
| | | | (G894T) | | GluAsp | Subnormal |
| | | | | | GluGlu | Normal |
| | NPY | 7p15.1 | L 7 P | rs16139 | LeuLeu | Normal |
| | | | | | LeuPro | Subnormal |
| | | | | | ProPro | deficient |
| | PPARA | 22q13.31 | L162V | rs1800206 | LL | Subnormal |
| | | | | | LV | Normal |
| | | | | | VV | Subnormal |
| | TNF | 6p21.3 | G-308A/TNF2 | rs1800629 | AA | Deficient |
| | | | | | GA | Subnormal |
| | | | | | GG | Normal |

In another embodiment, the present invention provides a method for determining genetic predisposition for deficiency in DNA methylation of a person. Similar to the method described above for determining genetic predisposition for deficiency in glycation, a panel of methylation identifier SNPs have been identified and provided in Table 3, expressed by their gene symbols, gene locations, polymorphism names, dbSNP ID, and the genotypes.

TABLE 3

| | | Gene | Polymorphism | | | |
|---|---|---|---|---|---|---|
| Gene Class | Gene Symbol | Location | Name | dbSNP_ID | Genotype | Rating |
| Methylation | APOC3 | 11q23.1-q23.2 | T-2854G | rs2542051 | TT | Normal |
| | | | | gs47114 | TG | Subnormal |
| | | | | | GG | Deficient |
| | CETP | 16q21 | Ile405Val | rs5882 | IleIle | Deficient |
| | | | | | IleVal | Subnormal |
| | | | | | ValVal | Normal |
| | | | TaqlB | rs708272 | B1B1 | Deficient |
| | | | | | B1B2 | Deficient |
| | | | | | B2B2 | Normal |
| | MTHFR | 1p36.3 | A1298C | rs1801131 | AA | Normal |
| | | | | | AC | Subnormal |
| | | | | | CC | Deficient |
| | | | C677T | rs1801133 | CC | Subnormal |
| | | | | | CT | Normal |
| | | | | | TT | Subnormal |

As shown, in this case a panel of five SNPs in three genes are used. These genes are apolipoprotein C-III (APOC3), cholesteryl ester transfer protein, plasma (CETP), and 5,10-methylenetetrahydrofolate reductase (NADPH) (MTHFR). Same to that described above, individual rating, overall rating or weighted overall rating are used for determining genetic predisposition for deficiency in DNA methylation of a person.

In a further embodiment, the result of determination of the genetic predisposition in one health function described above is further used in determination of the genetic predisposition in another health function. For example, a person having a genetic predisposition for deficiency in inflammation, as determined using the above-described panel of inflammation identifier SNPs, also tends to have deficiency in DNA methylation. Therefore, the determinations of the genetic predisposition for DNA methylation and inflammation can be mutually related. In one embodiment, in the determination of genetic predisposition for DNA methylation, the overall rating or weighted overall rating from the determination of genetic predisposition for inflammation is also used in obtaining the overall rating or weighted overall rating for DNA methylation. In another embodiment, the overall rating or weighted overall rating from the determination of genetic predisposition for DNA methylation is used in obtaining the overall rating or weighted overall rating for inflammation.

In a further aspect of the present invention, the method further includes providing a supplement composition to a person who has been identified to have genetic predisposition for deficiency in DNA methylation, for use as a dietary supplement to specifically enhance the person's DNA methylation and decrease the likelihood of developing cardiovascular diseases. A supplement composition specific for improving DNA methylation and the method of use have been described in detail in a co-pending Provisional Paten Application Ser. No. 60/702,057, entitled “Supplement Composition and Method of Use in Enhancement of an Individual's Methylation Process”, which is herein incorporated by reference in its entirety. Such a supplement composition is particularly suitable for the purpose of the invention.

In yet a further embodiment, the present invention provides a method for determining genetic predisposition for deficiency in oxidation process of a person. Similar to the method described above for determining genetic predisposition for deficiency in glycation, a panel of oxidation identifier SNPs have been identified and provided in Table 4, expressed by their gene symbols, gene locations, polymorphism names, dbSNP ID, and the genotypes.

As shown, in this case a panel of seven SNPs in six genes are used. These genes are advanced glycosylation end product-specific receptor (AGER), catalase (CAT), cytochrome P450, family 2, subfamily D, polypeptide 6 (CYP2D6), NAD(P)H dehydrogenase, quinone 1 (NQO1), superoxide dismutase 2, mitochondrial (SOD2), and superoxide dismutase 3, extracellular (SOD3). Same to that described above, individual rating, overall rating or weighted overall rating are used for determining genetic predisposition for deficiency in oxidation process of a person.

TABLE 4

| | | Gene | Polymorphism | | | |
|---|---|---|---|---|---|---|
| Gene Class | Gene Symbol | Location | Name | dbSNP_ID | Genotype | Rating |
| Oxidation | AGER | 6p21.3 | A2184G | rs3134940 | AA | Normal |
| | | | | | AG | Subnormal |
| | | | | | GG | Deficient |
| | | | Gly82Ser | rs2070600 | GlyGly | Normal |
| | | | | | GlySer | Subnormal |
| | | | | | SerSer | Deficient |
| | CAT | 11p13 | C(-262)T | rs1001179 | CC | Deficient |
| | | | | | CT | Subnormal |
| | | | | | TT | Normal |
| | CYP2D6 | 22q13.1 | C100T | rs1065852 | TT | Deficient |
| | | | | | TC | Subnormal |
| | | | | | CC | Normal |
| | NQO1 | 16q22.1 | Pro187Ser | rs1800566 | ProPro | normal |
| | | | C609T | | ProSer | Subnormal |
| | | | | | SerSer | Deficient |
| | SOD2 | 6q25.3 | Ala-9Val | rs1799725 | AlaAla | Normal |
| | | | | | AlaVal | Subnormal |
| | | | | | ValVal | Deficient |
| | SOD3 | 4p16.3-q21 | Arg213Gly | rs1799895 | ArgArg | Deficient |
| | | | | | ArgGly | Subnormal |
| | | | | | GlyGly | Normal |

In a further aspect of this embodiment, the method further includes providing a supplement composition to a person who has been identified having genetic predisposition for deficiency in oxidation, or more prone to oxidative damages, for use as a dietary supplement to specifically improve the person's resistance to oxidative damages and decrease the likelihood of developing clinical conditions directly or indirectly caused by oxidative damages. A supplement composition specific for improving a person's resistance to oxidative damages and the method of use have been described in detail in a co-pending Provisional Patent Application Ser. No. 60/685,142, entitled “Antioxidant Composition and Method of Use”, which is herein incorporated by reference in its entirety. Such a supplement composition is particularly suitable for the purpose of the invention.

In another embodiment, the present invention provides a method for determining genetic predisposition for deficiency in DNA repair of a person. Similar to the method described above for determining genetic predisposition for deficiency in glycation, a panel of DNA repair identifier SNPs have been identified and provided in Table 5, expressed by their gene symbols, gene locations, polymorphism names, dbSNP ID, and the genotypes.

As shown, in this case a panel of six SNPs in four genes are used. These genes are excision repair cross-complementing rodent repair deficiency, complementation group 2 (xeroderma pigmentosum D) (ERCC2), 8-oxoguanine DNA glycosylase (OGG1), poly (ADP-ribose) polymerase family, member 1 (PARP1), ADP-ribosyltransferase (NAD+; poly (ADP-ribose) polymerase)-like 4 (ADPRTL4), and X-ray repair complementing defective repair in Chinese hamster cells 1 (XRCC1). Same to that described above, individual rating, overall rating or weighted overall rating are used for determining genetic predisposition for deficiency in DNA repair of a person.

TABLE 5

| | | Gene | Polymorphism | | | |
|---|---|---|---|---|---|---|
| Gene Class | Gene Symbol | Location | Name | dbSNP_ID | Genotype | Rating |
| DNA repair | ERCC2 | 19q13.3 | Asp 312 Asn | rs1799793 | AsnAsn | Deficient |
| | | | | | AsnAsp | Subnormal |
| | | | | | AspAsp | Normal |
| | | | Lys 751 Gln | rs13181 | GlnGln | Deficient |
| | | | | | LysGln | Subnormal |
| | | | | | LysLys | Normal |
| | OGG1 | 3p26.2 | Ser326Cys | rs1052133 | CysCys | Deficient |
| | | | | | SerCys | subnormal |
| | | | | | SerSer | Normal |
| | PARP1/ADPRT | 1q41-q42 | Val762Ala | rs1136410 | AlaAla | Subnormal |
| | | | | | ValAla | Subnormal |
| | | | | | ValVal | Normal |
| | XRCC1 | 19q13.2 | Arg280His | rs25489 | ArgArg | Normal |
| | | | | | ArgHis | Subnormal |
| | | | | | HisHis | Deficient |
| | | | Arg399Gln | rs25487 | ArgArg | Normal |
| | | | | | ArgGln | Deficient |
| | | | | | GlnGln | Deficient |

In a further aspect of this embodiment, the method further includes providing a supplement composition to a person who has been identified having a genetic predisposition for deficiency in DNA repair, for use as a dietary supplement to specifically improve the person's DNA repair capability, decrease the likelihood of developing clinical conditions, such as cancers, directly or indirectly caused by deficient DNA repair. A supplement composition specific for improving a person's DNA repair and the method of use have been described in detail in a co-pending Provisional Patent Application Ser. No. 60/685,143, entitled “Supplement Composition and Method of Use for Enhancement of DNA Repair Process”, which is herein incorporated by reference in its entirety. Such a supplement composition is particularly suitable for the purpose of the invention.

The invention has been described with reference to particularly preferred embodiments. It will be appreciated, however, that various changes can be made without departing from the spirit of the invention, and such changes are intended to fall within the scope of the appended claims. While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents. All patents and other publications cited herein are expressly incorporated by reference.

What is claimed is:

1. A method of determining genetic predisposition for deficiency in a health function of a person using single nucleotide polymorphism (SNP) analysis comprising the steps of:
(a) obtaining a sample from a person;
(b) performing a SNP genotyping assay of three or more genes using said sample;
(c) obtaining a SNP panel comprising predetermined identifier SNPs;
(d) comparing said SNP panel with a predetermined criterion defining said genetic predisposition for deficiency in said health function; and
(e) reporting presence of said genetic predisposition for deficiency in said health function if said SNP panel meets said predetermined criterion.

2. The method of claim 1, wherein said health function is one selected from the group consisting of glycation, inflammation, DNA methylation, oxidation and DNA repair.

3. The method of claim 1 further comprising providing to said person a nutritional supplement specific to improve said health function if said genetic predisposition for deficiency in said health function is identified.

4. A method of determining genetic predisposition for deficiency in glycation of a person using single nucleotide polymorphism (SNP) analysis comprising the steps of:
(a) obtaining a sample from a person;
(b) performing a SNP genotyping assay of three or more genes using said sample;
(c) obtaining a glycation SNP panel comprising predetermined glycation identifier SNPs;
(d) comparing said glycation SNP panel with a predetermined criterion defining said genetic predisposition for deficiency in glycation; and
(e) reporting presence of said genetic predisposition for deficiency in glycation if said glycation SNP panel meets said predetermined criterion.

5. The method of claim 4, wherein said genes include ACE, LEPR, and PPARG.

6. The method of claim 4, wherein said predetermined glycation identifier SNPs include rs4646994, rs1137101, rs1137100, and rs3856806, expressed by dbSNP ID.

7. The method of claim 4 further comprising providing to said person a nutritional supplement specific to reduce said glycation if said genetic predisposition for deficiency in glycation is identified.

8. A method of determining genetic predisposition for deficiency in inflammation of a person using single nucleotide polymorphism (SNP) analysis comprising the steps of:
(a) obtaining a sample from a person;
(b) performing a SNP genotyping assay of three or more genes using said sample;
(c) obtaining an inflammation SNP panel comprising predetermined inflammation identifier SNPS;
(d) comparing said inflammation SNP panel with a predetermined criterion defining said genetic predisposition for deficiency in inflammation; and
(e) reporting presence of said genetic predisposition for deficiency in inflammation if said inflammation SNP panel meets said predetermined criterion.

9. The method of claim 8, wherein said genes include ICAM1, IL6, NFKB1, NOS3, NPY, PPARA, and TNF.

10. The method of claim 8, wherein said predetermined inflammation identifier SNPs include rs5498, rs1800795, rs28362491, rs1799983, rs16139, rs1800206, and rs1800629, expressed by dbSNP ID.

11. The method of claim 8 further comprising providing to said person a nutritional supplement specific to decrease a likelihood in developing said inflammation if said genetic predisposition for deficiency in inflammation is identified.

12. A method of determining genetic predisposition for deficiency in DNA methylation of a person using single nucleotide polymorphism (SNP) analysis comprising the steps of:
(a) obtaining a sample from a person;
(b) performing a SNP genotyping assay of three or more genes using said sample;
(c) obtaining a methylation SNP panel comprising predetermined methylation identifier SNPs;
(d) comparing said methylation SNP panel with a predetermined criterion defining said genetic predisposition for deficiency in DNA methylation; and
(e) reporting presence of said genetic predisposition for deficiency in DNA methylation if said methylation SNP panel meets said predetermined criterion.

13. The method of claim 12, wherein said genes include APOC3, CETP, and MTHFR.

14. The method of claim 12, wherein said predetermined methylation identifier SNPs include rs2542051, gs47114, rs5882, rs708272, rs1801131, and rs1801133, expressed by dbSNP ID.

15. The method of claim 12 further comprising providing to said person a nutritional supplement specific to improve said DNA methylation if said genetic predisposition for deficiency in DNA methylation is identified.

16. A method of determining genetic predisposition for deficiency in oxidation of a person using single nucleotide polymorphism (SNP) analysis comprising the steps of:
(a) obtaining a sample from a person;
(b) performing a SNP genotyping assay of three or more genes using said sample;
(c) obtaining an oxidation SNP panel comprising predetermined oxidation identifier SNPs;
(d) comparing said oxidation SNP panel with a predetermined criterion defining said genetic predisposition for deficiency in oxidation; and
(e) reporting presence of said genetic predisposition for deficiency in oxidation if said oxidation SNP panel meets said predetermined criterion.

17. The method of claim 16, wherein said genes include AGER, CAT, CYP2D6, NQO1, SOD2, and SOD3.

18. The method of claim 16, wherein said predetermined oxidation identifier SNPs include rs3134940, rs2070600, rs1001179, rs1065852, rs1800566, rs1799725, and rs1799895, expressed by dbSNP ID.

19. The method of claim 16 further comprising providing to said person a nutritional supplement specific to enhance resistance to oxidative damages if said genetic predisposition for deficiency in oxidation is identified.

20. A method of determining genetic predisposition for deficiency in DNA repair of a person using single nucleotide polymorphism (SNP) analysis comprising the steps of:
(a) obtaining a sample from a person;
(b) performing a SNP genotyping assay of three or more genes using said sample;
(c) obtaining a DNA repair SNP panel comprising predetermined DNA repair identifier SNPs;
(d) comparing said DNA repair SNP panel with a predetermined criterion defining said genetic predisposition for deficiency in DNA repair; and
(e) reporting presence of said genetic predisposition for deficiency in DNA repair if said DNA repair SNP panel meets said predetermined criterion.

21. The method of claim 20, wherein said genes include ERCC2, OGG1, PARP1/ADPRT, and XRCC1.

22. The method of claim 20, wherein said predetermined DNA repair identifier SNPs include rs1799793, rs13181, rs1052133, rs1136410, rs25489, and rs25487, expressed by dbSNP ID.

23. The method of claim 20 further comprising providing to said person a nutritional supplement specific to improve said DNA repair if said genetic predisposition for deficiency in DNA repair is identified.

24. A determinant single nucleotide polymorphism (SNP) panel for determining genetic predisposition for deficiency in health functions of a person comprising:
(a) a glycation SNP panel comprising predetermined glycation identifier SNPs;
(b) an inflammation SNP panel comprising predetermined inflammation identifier SNPs;
(c) a methylation SNP panel comprising predetermined methylation identifier SNPs;
(d) an oxidation SNP panel comprising predetermined oxidation identifier SNPs; and
(e) a DNA repair SNP panel comprising predetermined DNA repair identifier SNPs.

25. The determinant SNP panel of claim 24, wherein said predetermined glycation identifier SNPs include rs4646994, rs1137101, rs1137100, and rs3856806, expressed by dbSNP ID.

26. The determinant SNP panel of claim 24, wherein said predetermined inflammation identifier SNPs include rs5498, rs1800795, rs28362491, rs1799983, rs16139, rs1800206, and rs1800629, expressed by dbSNP ID.

27. The determinant SNP panel of claim 24, wherein said predetermined methylation identifier SNPs include rs2542051, gs47114, rs5882, rs708272, rs1801131, and rs1801133, expressed by dbSNP ID.

28. The determinant SNP panel of claim 24, wherein said predetermined oxidation identifier SNPs include rs3134940, rs2070600, rs1001179, rs1065852, rs1800566, rs1799725, and rs1799895, expressed by dbSNP ID.

29. The determinant SNP panel of claim 24, wherein said predetermined DNA repair identifier SNPs include rs1799793, rs13181, rs1052133, rs1136410, rs25489, and rs25487, expressed by dbSNP ID.
